(19) United States
(12) Patent Application Publication
(10) Pub. No.: US 2022/0211600 A1
(43) Pub. Date: Jul. 7, 2022

(54) ISOTROPIC LIQUID CLEANSERS COMPRISING ACYL ISETHIONATE AND METHYL ACYL TAURATE SURFACTANT MIXTURES

(71) Applicant: Conopco, Inc., d/b/a UNILEVER, Englewood Cliffs, NJ (US)

(72) Inventors: Douglas John HIBAN, Shelton, CT (US); Jamie Lynn MILLER, North Haven, CT (US); Tirucheral Varahan VASUDEVAN, Bethany, CT (US)

(73) Assignee: Conopco, Inc., d/b/a/ UNILEVER, Englewood Cliffs, NJ (US)

(21) Appl. No.: 17/611,705

(22) PCT Filed: May 4, 2020
(86) PCT No.: PCT/EP2020/062328

§ 371 Date: Nov. 16, 2021

(30) Foreign Application Priority Data
May 21, 2019 (EP) 19175538.8

(51) Int. Cl. A61K 8/46; A61Q 5/02; A61Q 19/10

(52) U.S. Cl. CPC A61K 8/466; A61K 2800/596; A61Q 19/10; A61Q 5/02

(57) ABSTRACT

The invention relates to isotropic liquid composition comprising acyl isethionate, methyl acyl taurate and amphoteric, zwitterionic and/or nonionic surfactant. Unexpectedly, it has been found that, when ratio of isethionate to taurate is monitored, enhanced lather is achieved.

### FIELD OF THE INVENTION

The present invention relates to liquid cleansing compositions suitable for topical application for cleansing the human body, such as skin and hair. In particular, the invention relates to compositions which, at least in one embodiment, are preferably sulfate free and mild to the skin and scalp. The compositions preferably are able to lather appreciably, are stable and have a micellar (isotropic) microstructure.

### BACKGROUND OF THE INVENTION

Consumers seek sulfate free personal cleansing compositions (e.g., having no sulfate-based surfactants) that are extremely mild and moisturizing while delivering superior sensory benefits such as creamy lather and soft, smooth skin, preferably after one shower. Acyl isethionates are known to be extremely mild surfactants and are an ideal surfactant for delivering mildness and moisturization with voluminous and creamy lather that consumers desire. However, liquid cleansers containing high levels of acyl isethionates tend to crystallize due to the low solubility of acyl isethionates in aqueous systems.

Sun et al (Journal of Cosmetic Science, 54, 559-568, 2003) have suggested several surfactants that can be used to solubilize acyl isethionates such as methyl acyl taurates, acyl glutamates, acyl lactylates, alkyl ether and dialkyl sulfosuccinates, and acyl sarcosinates. Among the surfactants outlined, methyl acyl taurate offers a distinct advantage over the others in that methyl acyl taurates and acyl isethionates can commercially be synthesized as a mixture in a single pot reactor (U.S. Pat. No. 6,562,874 to Ilardi et al.).

In the present invention, unexpectedly, it has been found that a mixture of acyl isethionate and methyl acyl taurate, when combined in a specific weight ratio, exhibit synergy and generate enhanced lather volume, while maintaining lather creaminess, attributes which are desired by consumers.

U.S. Pat. No. 5,415,810 to Lee et al. discloses acyl isethionate containing liquids that also contain other anionic surfactants such as methyl acyl taurates, but there is no recognition of any synergy (e.g., in lather) between acyl isethionates and methyl acyl taurates used in a specific range of ratio between the two surfactants.

U.S. Pat. No. 5,925,603 to D'Angelo discloses the use of methyl acyl taurate as a solubilizing surfactant for acyl isethionates, but there is no recognition that specific ratios of acyl isethionate to methyl acyl taurate can produce synergistic lather performance. The disclosed compositions are also stipulated to be between 7.5 and 8.5 pH values. At these pH values, acyl isethionates can undergo hydrolysis when held at the type of higher temperatures that are quite prevalent in tropical regions. It is desirable to formulate acyl isethionate formulations between pH values from 6 to 7.3 to avoid hydrolysis. Formulations of the subject invention have a pH of 5.0 to 7.4, including all ranges subsumed therein, and preferably, 6.0 to 7.3. In an embodiment of the invention, formulation pH is from 6.3 to 7.3, including all ranges subsumed therein.

US 2009/0062406 A1 to Loeffler et al. discloses flowable aqueous concentrates comprising a mixture of acyl isethionate, methyl acyl taurate and alkyl betaines. There is no recognition that specific ratios between acyl isethionate and methyl acyl taurate can be synergistic in terms of generating consumer desired lather. Compositions of the present invention have surfactant levels below 20% by weight of the composition. In one embodiment, the total surfactant level of the compositions of the present invention is from 2 to 15% by weight and in another embodiment, the total surfactant level is from 3 to less than 15% by weight, including all ranges subsumed therein. In still another embodiment, the surfactant level of the compositions of the present invention is from 4 to 15% by weight, including all ranges subsumed therein.

A lamellar liquid crystalline composition comprising of mixtures of isethionate surfactants, taurate surfactants and sarcosinate surfactants is disclosed in U.S. Pat. No. 9,187,716 to Griffin et al. containing at least 2% by weight of an electrolyte. No mention or implication of any synergy between methyl acyl taurate and acyl isethionate is evident from this invention. Further, the amount of isethionate is greater than 3 times the amount of taurate in the examples (Examples 1-A, 2-A). Finally, the compositions are lamellar, not isotropic as those described in the claimed invention.

US 2017/0304173 to Elder et al. discloses compositions for make-up removal that comprise mixtures of acyl isethionate and methyl acyl taurates. Again, there is no recognition that specific ratios between acyl isethionate and methyl acyl taurate can be synergistic in terms of generating consumer desired lather. Additionally, the disclosed compositions in the reference require the use of non-ionic emulsifiers which are efficacious for removing make up.

However, use of non-ionic emulsifiers is optional in the mild skin and hair cleansing compositions of the present invention. In an embodiment of the invention, compositions (or formulations) are substantially free of nonionic emulsifiers (or surfactants) where substantially free means less than 0.01 percent by weight based on total weight of the composition. In another embodiment, the composition of the present invention comprises less than 0.008% and in still another embodiment less than 0.005% by weight nonionic emulsifier based on total weight of the composition. In still another embodiment, the composition of the present invention comprises from 0.00001 to 0.004% by weight nonionic emulsifier. In yet another embodiment, the composition is free of (0.0%) nonionic emulsifier. In still another embodiment, the composition of this invention is substantially free of nonionic emulsifiers when amphoteric surfactant (including betaines), zwitterionic surfactant or a mixture thereof are present at 0.1% by weight or more of the composition. When betaine, zwitterionic and/or amphoteric surfactant (including betaines) do not exceed 0.1% by weight of the composition, it is within the scope of the invention for the composition to comprise from 0.00001 to 8% by weight nonionic surfactant, including all ranges subsumed therein, and in another embodiment, from 0.001 to 7% by weight nonionic surfactant and in still another embodiment, from 1 to 6% by weight nonionic surfactant.

Unexpectedly, it has been found that when the ratio of acyl isethionate to methyl acyl taurate is tightly controlled (1.5:1 to 1:1.5), a significant boost in lather volume is obtained (from about 300 mL, measured at 45 seconds to about 630 mL measured at 45 seconds) compared to that obtained at the same level (as mixture) of individual surfactants.

### SUMMARY OF THE INVENTION

The present invention is directed to an isotropic personal cleansing liquid composition. Isotropic compositions are those in which surfactant micelles do not tend to aggregate and form lamellar (liquid crystalline) layers. The composition comprises:
1) 0.1 to 8%, preferably 0.5 to 6%, more preferably 1 to 4% by weight. of acyl isethionate;
2) 0.1% to 8%, preferably 0.5 to 6%, more preferably 1 to 4% by weight of methyl acyl taurate;
3) 0.0 to 15%, preferably 0.1 to 10% and more preferably 0.5 to 8% by wt. of an amphoteric and/or zwitterionic surfactant; and
4) 0.0 to 8.0%, preferably 0.001 to 7%, and more preferably, 1 to 6% by weight nonionic surfactant,

with the proviso that sum of all surfactant is less than 20% by weight and the composition does not simultaneously comprise 0.01% by weight or more nonionic surfactant and 0.1% by weight or more amphoteric surfactant (including betaine) and/or zwitterionic surfactant and does not simultaneously comprise 0.0% by weight amphoteric and/or zwitterionic surfactant and 0.0% by weight nonionic surfactant, wherein the ratio of acyl isethionate to methyl acyl taurate surfactant is between 1.5:1 to 1:1.5, and more preferably, from 1.25:1 to 1:1.25, and preferably, 1.1:1 to 1:1.1, and most preferably, 1:1.

In an embodiment of the invention, the ratio of amphoteric, zwitterionic and/or nonionic to anionic is 1:1 to 4:1, preferably 1:1 to 3:1, and most preferably, from 1.8:1 to 2.2:1 with the proviso that sum of all the surfactants is less than 20% by weight and that the composition pH is 5.0 to 7.4, preferably 6.0 to 7.3 and further wherein the composition is substantially free of nonionic surfactant. In some embodiments, pH is 6.3 to 7.3, including all ranges subsumed therein. In an especially preferred embodiment, the ratio of amphoteric to anionic is 2:1.

In still another embodiment of the invention amphoteric and/or zwitterionic surfactant make(s) up from 0.0 to less than 0.1% by weight of the composition and nonionic surfactant is present at an amount from 0.02 to 8% by weight of the composition.

### DETAILED DESCRIPTION OF THE INVENTION

Except in the examples, or where otherwise explicitly indicated, all numbers in this description indicating amounts of material or conditions of reaction, physical properties of materials and/or use are to be understood as modified by the word “about.”

As used throughout, ranges are used as shorthand for describing each and every value that is within the range, and therefore, all ranges include the values subsumed therein unless otherwise stated. Any value within the range can be selected as terminus of the range. The use of and/or indicates that any one from the list can be chosen individually, or any combination from the list can be chosen.

For the avoidance of doubt, the word “comprising” is intended to mean “including” but not necessarily “consisting of” or “composed of.” In other words, the listed steps or options need not be exhaustive.

Unless indicated otherwise, all percentages for amount or amounts of ingredients used are to be understood to be percentages by weight based on the active weight of the material in the total weight of the composition, which total is 100%. Emulsifier and surfactant can be used interchangeably herein. For the avoidance of doubt, amphoteric surfactants include betaines. Nonionic surfactant, as used herein, includes amine oxides.

The invention relates to isotropic liquid cleansing compositions comprising acyl isethionate, methyl acyl taurate, and at least one of, an amphoteric, and/or nonionic surfactant. Isotropic compositions are those in which the surfactants form micelles but do not aggregate to form lamellar (liquid crystalline) layers. The invention relates to isotropic cleansing compositions comprising less than 0.2% by weight sulfate based surfactant. In another embodiment, the invention is directed to a composition comprising from 0.0001 to less than 0.2% by weight sulfate based surfactant. In still another embodiment, the invention is directed to a composition having no (0.0% by weight) sulfate based surfactant. In still another embodiment of the invention, the composition comprises less than 3% by weight betaine and yet in another embodiment no (0.0% by weight) betaine.

When the acyl isethionate and acyl methyl taurate are kept within a ratio of 1.5:1 to 1:1.5; the overall level of surfactants is kept at less than 20% by weight of the composition; and the pH is at 5.0 to 7.4, preferably 6.0 to 7.3, the benefits of mildness are produced and maintained by the composition of the present invention while significantly enhancing lather compared to compositions where ratio of acyl isethionate to methyl acyl taurate is outside these ranges. The compositions may optionally comprise amphoteric, zwitterionic and/or nonionic surfactant and the ratio of such surfactant to anionic surfactant can be 1:1 and higher.

More specifically, the invention comprises:
1) 0.1 to 8%, preferably 0.5 to 6%, more preferably 1 to 4% by wt. of acyl isethionate; 2) 0.1% to 8%, preferably 0.5 to 6%, more preferably 1 to 4% by weight of methyl acyl taurate;
3) 0.0 to 15%, preferably 0.5 to 10% and more preferably 1 to 8% by wt. of an amphoteric and/or zwitterionic surfactant; and
4) 0.0 to 8%, preferably, 0.001 to 7%, more preferably, 1 to 6% by weight nonionic surfactant

with the proviso that the sum of all surfactant is less than 20% by weight and the composition does not simultaneously comprise 0.01% by weight or more nonionic surfactant and 0.1% by weight or more amphoteric surfactant (including betaine) and/or zwitterionic surfactant and does not simultaneously comprise 0.0% by weight amphoteric and/or zwitterionic surfactant and 0.0% by weight nonionic surfactant, wherein the ratio of acyl isethionate to methyl acyl taurate surfactant is between 1.5:1 to 1:1.5, and more preferably, from 1.25:1 to 1:1.25, and preferably, 1.1:1 to 1:1.1, and most preferably, 1:1 and further wherein ratio of item (3) and/or item (4) to anionic surfactant (e.g., components (1) and (2)) is 1:1, preferably. 2:1, more preferably, 3:1, and most preferably, 4:1 with the further proviso that the pH of the composition is 5.0 to 7.4, preferably 6.0 to 7.3.

The invention is described in more detail below.

The composition can comprise 0.1 to 8% by wt., preferably 0.5 to 6%, and more preferably 1 to 4% by wt. acyl isethionate.

Fatty acyl isethionates molecules (e.g., cocoyl isethionates) are anionic surfactants highly desirable in personal care skin or hair cleansing products, particularly in personal care products, because they lather well, are mild to the skin and have good emollient properties. Typically, fatty acyl isethionates are produced by direct esterification of fatty acids or by reaction of fatty acid chloride having carbon chain length of C8 to C20 with isethionate. A typical fatty acyl isethionate surfactant “product” (e.g., commercially sold or made surfactant product) contains about 40 to 95 wt. % of the fatty acyl isethionate product and 0 to 50 wt. % typically 5 to 40 wt. % free fatty acid, in addition to isethionate salts, typically at less than 5%, and trace (less than 2 wt. %) of other impurities.

A second required component of the claimed invention is methyl acyl taurate. This is present at a level of 0.1 to 8% by wt., preferably 0.5 to 6% and more preferably 1 to 4% by wt. methyl acyl taurates.

Methyl acyl taurates (or taurides) are a group of mild anionic surfactants. They are composed of a hydrophilic head group, consisting of N-methyltaurine (2-methylaminoethanesulfonic acid) and a lipophilic residue, consisting of a long-chain carboxylic acid (fatty acid), both linked via an amide bond. The fatty acids used could be lauric (C12), myristic (C14), palmitic (C16) or stearic acid (C18), but mainly mixtures of oleic acid (C18:1) and coconut fatty acid (C8-C18) are used. Besides sodium, no other counterions typically play a relevant role (other counterions could be e.g., ammonium or other alkali or alkaline earth metals).

According to the invention, as seen in the examples, when the ratio of methyl acyl taurate to acyl isethionate is 1.5:1 to 1:1.5, preferably 1.25:1 to 1:1.25 and particularly, 1:1 (and other requirements noted are met), the lather volumes are enhanced.

For example, at 12% total active level, as seen in Table 1.1 (e.g., 12% total surfactant, (amphoteric, zwitterionic and/or anionic) where amphoteric is 8% and anionic acyl methyl taurate and acyl isethionate are 4% (2% each) total), the lather volume is greatest at a ratio of 1:1 taurate to isethionate. The increase is seen more clearly when dilution is 5.0 g/250 ml rather than 2.5 g/250 ml (since there is more product), but the trend is seen even at lower level of dilution.

At 9% total surfactant (Table 2 in Example), there is slightly less anionic (6% to 3% anionic), so the lather volume is slightly lower at the same 5.0 g/250 ml dilution, but clearly lather is greater as the ratio approaches 1:1.

In Table 3, the levels of anionic are at 2% (4% amphoteric to 2% anionic), so, although the same trend is seen (best lather as we approach 1:1 ratio), more product (for example 10 g/250 ml) needs to be diluted to show the full effect. At dilution of 2.5 g/250 ml, the product level is too low since there is not lather of even 300 ml, and, accordingly, we have not recorded it.

Levels of lather, for purposes of the invention, should be at least of 200 ml, preferably at least 300 ml, when measured at 45 seconds according to the methodology described in the Sita® foam tester below.

As indicated above, the invention relates to compositions in which the ratio of the acyl isethionate to methyl acyl taurate is 1.5:1 to 1:1.5, preferably 1.25:1 to 1:1.25 and most preferably 1:1.

A third component of the invention is zwitterionic or amphoteric, preferably amphoteric surfactant.

Amphoteric surfactants which may be used in this invention include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula:

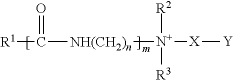

where R¹ is alkyl or alkenyl of 7 to 8 carbon atoms;

R² and R³ are each independently alkyl, hydroxyalkyl or carboxyalkyl of 1 to 3 carbon atoms;

n is 2 to 4;

m is 0 to 1;

X is alkylene of 1 to 3 carbon atoms optionally substituted with hydroxyl, and

Y is —CO₂— or —SO₃—.

In one embodiment, amphoteric may be alkylamido alkyl betaine (e.g. cocoamidopropyl betaine). It may also be an amphoacetate; or a hydroxy sultaine (e.g., cocoamidopropyl hydroxy sultaine).

Zwitterionic surfactants are exemplified by those which can be broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy sulfonate, sulfate, phosphate, or phosphonate. A general formula for these compounds is:

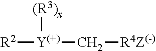

wherein R² contains an alkyl, alkenyl, or hydroxy alkyl radical of from about 8 to about 18 carbon atoms, from 0 to about 10 ethylene oxide moieties and from 0 to about 1 glycerol moiety; Y is selected from the group consisting of nitrogen, phosphorus and sulfur atoms; R³ is an alky or monohydroxylakyl group containing about 1 to about 3 carbon atoms; X is 1 when Y is a sulfur atom, and 2 when Y is a nitrogen or phosphorous atom; R⁴ is an alkylene or hydroxyalkylene of from about 1 to about 4 carbon atoms and Z is a radical selected from the group consisting of carboxylate, sulfonate, sulfate, phosphonate, and phosphate groups.

A fourth component of the invention is nonionic surfactant.

The nonionic which may be used includes, in particular, the reaction products of compounds having a hydrophobic group and a reactive hydrogen atom, for example aliphatic alcohols, acids, amides or alkyl phenols with alkylene oxides, especially ethylene oxide either alone or with propylene oxide. Specific nonionic detergent compounds are alkyl (C.sub.6-C.sub.22) phenols-ethylene oxide condensates, the condensation products of aliphatic (C.sub.8-C.sub.18) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxides.

In an embodiment of the invention, the nonionic surfactants used in this invention include lauryl amidopropyl amine oxide, lauramine oxide, cocoamidopropyl amine oxide or mixtures thereof. Such amine oxides are made commercially available from suppliers like Stepan under the Ammonyx name.

In compositions of the invention, the ratio of amphoteric, zwitterionic and/or nonionic surfactant to anionic surfactant is 1:1 to 4:1, preferably 1:1 to 3:1, more preferably, 1.8:1 to 2.2:1.

The liquid compositions may include a variety of other ingredients which are typically found in liquid cleanser compositions.

In addition to the specific isethionate, taurate, and amphoteric, zwitterionic and/or nonionic surfactant, the compositions may comprise small amounts of additional surfactants (typically used in an amount less than any of the three surfactants) as long as total amount of all surfactants is less than 20% by wt. of the liquid cleansing composition of the invention.

Other surfactants that may optionally be included are cationic surfactants as described in U.S. Pat. No. 3,723,325 to Parran Jr. and “Surface Active Agents and Detergents” (Vol. I & II) by Schwartz, Perry & Berch, both of which are incorporated into the subject application by reference.

Water soluble/dispersible polymers are an optional ingredient that is preferred to be included in the liquid composition of the invention. The water soluble/or dispersible polymers can be cationic, anionic, amphoteric or nonionic polymers with molecular weight higher than 100,000 Daltons. These polymers are known to enhance in-use and after-use skin sensory feel, to enhance lather creaminess and lather stability, and to increase the viscosity of liquid cleanser compositions.

Examples of water soluble/or dispersible structuring polymers useful in the present invention include the carbohydrate gums such as cellulose gum, microcrystalline cellulose, cellulose gel, hydroxyethyl cellulose, hydroxypropyl cellulose, sodium carboxymethylcellulose, tapioca starch, citrus fibers, hydroxymethyl or carboxymethyl cellulose, methyl cellulose, ethyl cellulose, guar gum, gum karaya, gum tragacanth, gum Arabic, gum acacia, gum agar, xanthan gum and mixtures thereof; modified and nonmodified starch granules with gelatinization temperature between 30 to 85° C. and pregelatinized cold water soluble starch; polyacrylate; Carbopols; alkaline soluble emulsion polymer such as Aculyn 28, Acuyln 22 or Carbopol Aqua SF1; cationic polymer such as modified polysaccharides including cationic guar available from Rhone Poulenc under the trade name Jaguar 013S, Jaguar 014S, Jaguar C17, or Jaguar C16; cationic modified cellulose such as UCARE Polymer JR 30 or JR 40 from Amerchol; N-Hance 3000, N-Hance 3196, N-Hance GPX 215 or N-Hance GPX 196 from Hercules; synthetic cationic polymer such as MerQuat 100, MerQuat 280, Merquat 281 and Merquat 550 by Nalco; cationic starches, e.g., StaLok® 100, 200, 300 and 400 made by Galactasol 800 series by Henkel, Inc.; Quadrosoft Um-200; and Polyquaternium-24. Preferably, the polymer comprises polysaccharide, polyacrylate or a mixture thereof, more preferably, the polymer is polysaccharide, polyacrylate or a mixture thereof.

Gel forming polymers such as modified or non-modified starch granules, xanthan gum, Carbopol, alkaline-soluble emulsion polymers and cationic guar gum such as Jaguar C13S, and cationic modified cellulose such as UCARE Polymer JR 30 or JR 40 are particularly preferred for this invention.

Water Soluble Skin Benefit Agents

Water-soluble skin benefit agents are another optional ingredient that is preferred to be included in the liquid compositions of the invention. A variety of water-soluble skin benefit agents can be used, and the level can be from 0 to 40 weight %, preferably 1 to 30%. The materials include, but are not limited to, polyhydroxy alcohols such as glycerol, propylene glycol, sorbitol, panthenol and sugar; urea, alpha-hydroxy acid and its salt such as glycolic or lactic acid, and low molecular weight polyethylene glycols with molecular weight less than 20,000. Preferred water-soluble skin benefit agents for use in the liquid compositions are glycerol, sorbitol and propylene glycol.

The liquid cleansing composition of the invention also may comprise 0 to 40% by wt. benefit agent. In another embodiment from 0.01 to 15% by weight and in still another embodiment from 0.02 to 10% by weight benefit agent, based on total weight of the composition and including all ranges subsumed therein.

One class of ingredients are nutrients used to moisturize and strengthen, for example, the skin. These include:
a) vitamins such as vitamin A and E, and vitamin alkyl esters such as vitamin C alkyl esters;
b) lipids such as cholesterol, cholesterol esters, lanolin, ceramides, sucrose esters, and pseudo-ceramides;
c) liposome forming materials such as phospholipids and suitable amphophilic molecules having two long hydrocarbon chains;
d) essential fatty acids, poly unsaturated fatty acids, and sources of these materials;
e) triglycerides of unsaturated fatty acids such as sunflower oil, primrose oil avocado oil, almond oil;
f) vegetable butters formed from mixtures of saturated and unsaturated fatty acids such as Shea butter;
g) minerals such as sources of zinc, magnesium, and iron.

A second type of skin benefit agent is a skin conditioner used to provide a moisturized feel to the skin. Suitable skin conditioners include:
a) silicone oils, gums and modifications thereof such as linear and cyclic polydimethylsiloxanes, amino, alkyl, and alkyl aryl silicone oils;
b) hydrocarbons such as liquid paraffins, petrolatum, Vaseline, microcrystalline wax, ceresin, squalene, pristan, paraffin wax and mineral oil;
c) conditioning proteins such as milk proteins, silk proteins and glutens;
d) cationic polymers as conditioners which may be used include Quatrisoft LM-200 Polyquaternium-24, Merquat Plus 3330-Polyquaternium 30; and Jaguar® type conditioners;
e) humectants such as glycerol, sorbitol, and urea;
f) emollients such as esters of long chain fatty acids, such as isopropyl palmitate and cetyl lactate.

A third type of benefit agent is deep cleansing agents. These are defined here as ingredients that can either increase the sense of refreshment immediately after cleansing or can provide a sustained effect on skin problems that are associated with incomplete cleansing. Deep cleansing agents include:
a) antimicrobials such as 2-hydrozy-4,2′,4′-trichlorodiphenylether (DP300) 2,6-dimeth-4-hydroxychlorobenzene (PCMX),3,4,4′-trichlorocarbanilide (TCC), 3-trifluoromethyl-4,4′-dichlorocarbanilide (TFC), benzoyl peroxide, zinc slats, tea tree oil, b) anti-acne agents such as salicylic acid, lactic acid, glycolic acid, and citric acid, and benzoyl peroxide (also an antimicrobial agent),
c) oil control agents including sebum suppressants, modifiers such as silica, titanium dioxide, oil absorbers, such as micro sponges,
d) astringents including tannins, zinc and aluminum salts, plant extracts such as from green tea and Witch-hazel (Hammailes),
e) scrub and exfoliating particles, such as polyethylene spheres, agglomerated silica, sugar, ground pits, seeds, and husks such as from walnuts, peach, avocado, and oats, salts,
f) cooling agents such as methanol and its various derivatives and lower alcohols,
g) fruit and herbal extracts,
h) skin calming agent such as aloe vera,
i) essential oils such as mentha, jasmine, camphor, white cedar, bitter orange peel, rye, turpentine, cinnamon, bergamot, citrus unshiu, calamus, pine, lavender, bay, clove, hiba, eucalyptus, lemon, starflower, thyme, peppermint, rose, sage, menthol, cineole, sugenol, citral, citronelle, borneol, linalool, geraniol, evening primrose, camphor, tymol, spirantol, pinene, limonene and terpenoid oils.

Other benefits agents that can be employed include antiaging compounds, sunscreens, and skin lightening and benefit agents like vitamin B3, resorcinols (especially 4-substituted resorcinols like 4-ethyl- and 4-hexyl resorcinol), retinoids, as well as antibacterial agents including terpineol and/or thymol.

When the benefit agent is oil, especially low viscosity oil, it may be advantageous to pre-thicken it to enhance its delivery. In such cases, hydrophobic polymers of the type describe in U.S. Pat. No. 5,817,609 to He et al. may be employed, which is incorporated by reference into the subject application.

At ambient temperature, the composition contains surfactant crystals with dissolution temperature between 30° C. to 50° C. The compositions should also be physically phase stable at room temperature and 45° C. for at least two weeks.

Other Optional Components

In addition, the compositions of the invention may include 0 to 10% by wt. optional ingredients as follows:

Perfumes; sequestering agents, such as tetra sodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (styrene/acrylate copolymer); all of which are useful in enhancing the appearance of cosmetic properties of the product.

The compositions may further comprise antimicrobials such as 2-hydroxy-4,2′4′ trichlorodiphenyl ether (DP300); preservatives such as dimethylodimethylhydantoin (Glydant XL 1000), parabens, sorbic acid, phenoxyethanol, iodopropynyl butylcarbamate, mixtures thereof and the like. Such preservatives may be enhanced with well know preservative boosters such as 1,2-alkane diols, including 1,2-octane diol.

Antioxidants such as, for example, butylated hydroxytoluene (BHT) may be used advantageously in amounts of about 0.01% or higher if appropriate.

Polyethylene glycols as conditioners which may be used include:

| | | |
|---|---|---|
| Polyox | WSR-25 | PEG 14M, |
| Polyox | WSR-N-60K | PEG 45M, or |
| Polyox | WSR-N-750 | PEG 7M. |

Another ingredient which may be included are exfoliants such as polyoxyethylene beads, walnut shells and apricot seeds.

The invention further comprises method of preparing compositions comprising components (1), (2) and (3) of the compositions noted above, wherein the ratio of acyl isethionate to acyl methyl taurate is 1.5:1 to 1:1.5, preferably 1.25:1 to 1:1.25; wherein ratio of amphoteric/zwitterionic surfactant to anionic surfactant is 1:1 and higher; and wherein the sum of surfactants is less than 20% by wt., which method comprises:
1) mixing water and a structuring polymer to about 75° C. (70-80° C.);
2) adding isethionate, taurate and optional fatty acid and mixing until dissolved;
3) cooling to 55° C. and adding amphoteric, zwitterionic and/or nonionic; and
4) adjusting pH to 5.0 to 7.4.

The invention further comprises use of compositions of the invention to enhance lather.

Compositions of the invention are isotropic and an example of how to prepare is also discussed in the protocol.

### Examples and Protocol

In all the ensuring examples the lather was created using a Sita Foam apparatus and the procedure is shown below.

Various dilutions of product with water, ranging from 2.5 grams product to 250 grams of water to 10 grams of product to 250 grams of water since consumers would use a range of product amounts in the shower and the dilution used in these examples approximates that range. Additionally, consumers may also rub the products on the skin, either with hand or pouf, with different force and to approximate that some of the tests were run at two different stirrer speeds.

Sita Foam Tester R-2000 Procedure

The Sita Foam (Sita Foam Tester R-2000) was used to measure foam generated under a specified dilution and shear rate. It utilizes a rotor at high speeds which both mixes the product with dilution water and creates lather volume. The rotor creates a vortex, which incorporates air; lathering at different rates depending on the ability of the formulation. To operate the Sita Foam, the measurement parameters are into the application in the “Device” drop down menu. See the table below for the parameters used during these measurements.

| | | | Foam Build Up Measurement | | |
|---|---|---|---|---|---|
| | Series | Fill | Stir | Stir | |
| Parameter | Count | with | Counts | Time | Revolution |
| Medium | 1 | 250 mL | 4 | 15 s | 1000 min⁻¹ |
| Shear Test | | | | | |
| High Shear | 1 | 250 mL | 4 | 15 s | 1500 min⁻¹ |
| Test | | | | | |

A heat exchanger should be connected to the Sita Foam's glass vessel to ensure a consistent temperature throughout the testing. Set the heat exchanger to 38 Celsius, and wait 15 minutes for the temperature to reach 38° C. Fluctuation of the temperature in the heat exchanger between 37° C. and 39° C. is acceptable.

Dispense 1 g, 2.5 g, 5 g, or 10 g of product into the Sita Foam glass vessel, ensuring that the product does not land on the sides of the vessel or on the rotor, which can cause inaccuracy in the readings. Then add water to the holding tank in the back of the Sita Foam. Adjust the water temperature to between 37° C. and 39° C. This water will be used to dilute the product and generate lather.

Start the run on the Sita Foam. The Sita Foam will automatically dilute the product, then mix for 15 seconds. The run mixes four separate times, taking a measurement between each reading. At least three readings should be taken for each unique sample at each dilution desired.

Readings below 300 mL of foam generation are not to be considered due to susceptibility to error for reliable evaluation. If the standard error at 2.5 g and 5 g is too high to discern differentiation, increase product dosing to 10 g, or change parameters to those seen in the high shear test using 1 g of product.

### EXAMPLES

General isotropic formulations are set forth in Examples A to C below and Examples 1-7 highlight mixtures of surfactant systems used in the general formulations A to C.

### Example A

Isotropic (Compositions of Tables 1, 2, 3, 6 and 7) (Sodium Cocoyl Isethionate/Sodium Methyl Lauroyl Taurate)

| | |
|---|---|
| Chemical | Typical Range wt % |

| | | |
|---|---|---|
| DI Water | Q.S | Q.S |
| Synthalen W2000 | 0.60 | 0.60 |
| Sodium Cocoyl Isethionate | 0.00 | 4.00 |
| Sodium Methyl Lauroyl Taurate | 0.00 | 4.00 |
| Stearic Acid | 0.05 | 0.05 |
| Cocamidopropyl Betaine | 4.00 | 8.00 |
| Glycerin | 1.00 | 1.00 |
| Tetrasodium EDTA | 0.05 | 0.05 |
| Phenoxyethanol | 0.60 | 0.60 |
| Iodopropynyl Butylcarbamate | 0.07 | 0.07 |
| Sodium Hydroxide | 0.16 | 0.27 |
| PPG-7 | 0.00 | 0.96 |

### Example B

Isotropic (Compositions of Table 4) (Sodium Lauroyl Isethionate/Sodium Methyl Lauroyl Taurate)

| | |
|---|---|
| Chemical | Typical Range wt % |

| | | |
|---|---|---|
| DI Water | Q.S | Q.S |
| Synthalen W2000 | 0.60 | 0.60 |
| Sodium Lauroyl Isethionate | 0.00 | 3.00 |
| Sodium Methyl Lauroyl Taurate | 0.00 | 3.00 |
| Stearic Acid | 0.05 | 0.05 |
| Cocamidopropyl Betaine | 6.00 | 6.00 |
| Glycerin | 1.00 | 1.00 |
| Tetrasodium EDTA | 0.05 | 0.05 |
| Phenoxyethanol | 0.60 | 0.60 |
| Iodopropynyl Butylcarbamate | 0.07 | 0.07 |
| Sodium Hydroxide | 0.18 | 0.22 |
| Citric Acid | 0.00 | 0.03 |

### Example C

Isotropic (Compositions of Table 5) (Sodium Cocoyl Isethionate/Sodium Methyl Cocoyl Taurate)

| | |
|---|---|
| Chemical | Typical Range wt % |

| | | |
|---|---|---|
| DI Water | Q.S | Q.S |
| Synthalen W2000 | 0.60 | 0.60 |
| Sodium Cocoyl Isethionate | 0.00 | 3.00 |
| Sodium Methyl Cocoyl Taurate | 0.00 | 3.00 |
| Stearic Acid | 0.05 | 0.05 |
| Cocamidopropyl Betaine | 6.00 | 6.00 |
| Glycerin | 1.00 | 1.00 |
| Tetrasodium EDTA | 0.05 | 0.05 |
| Phenoxyethanol | 0.60 | 0.60 |
| Iodopropynyl Butylcarbamate | 0.07 | 0.07 |
| Sodium Hydroxide | 0.18 | 0.21 |
| Citric Acid | 0.00 | 0.03 |

TABLE 1
12% Surfactants with lauroyl methyl taurate and
cocoyl isethionate; 2:1 amphoteric to anionic

| Product Dilution | | | | |
|---|---|---|---|---|
| Lauroyl methyl | 5.0 g/250 mL, | | 2.5 g/250 mL, | |
| taurate/Cocoyl | 1000 RPM | | 1000 RPM | |
| Isethionate | Foam Volume | STD. | Foam Volume | STD. |
| (wt. %/wt. %) | (mL) at 45 Sec. | Error | (mL) at 45 Sec. | Error |
| 0.0%/100.0% | 549 | 16 | 343 | 8 |
| 50.0%/50.0% | 664 | 3 | 555 | 9 |
| 100.0%/0.0% | 569 | 8 | 534 | 4 |

Table 1: This formulation comprises 8% amphoteric (cocoamidopropyl betaine), 2% taurate, and 2% isethionate as set forth in model isotropic formula. Full formulations are as set forth in Examples A; full formulations are used for lather tests. The best synergy occurs at ratios of 1:1. Synergy of methyl acyl taurate to acyl isethionate exists at all tested dilutions of the 12% active body wash and the data indicates that criticality occurs at ratio of 1.5:1 to 1:1.5, preferably 1.25:1 to 1:1.25, and most preferably at 1:1 methyl acyl taurate to acyl isethionate ratio.

TABLE 2
Table 2: 9% Surfactants with lauroyl methyl taurate
and coocyl isethionate; 2:1 amphoteric to anionic

| Dilutions | | | | |
|---|---|---|---|---|
| Lauroyl methyl | 5.0 g/250 mL, | | 2.5 g/250 mL, | |
| taurate/Cocoyl | 1000 RPM | | 1000 RPM | |
| Isethionate | Foam Volume | STD. | Foam Volume | STD. |
| (wt. %/wt. %) | (mL) at 45 Sec. | Error | (mL) at 45 Sec. | Error |
| 0.0%/100.0% | 376 | 11 | 375 | 11 |
| 25.0%/75.0% | 447 | 18 | 374 | 6 |
| 40.0%/60.0% | 488 | 7 | 414 | 13 |
| 50.0%/50.0% | 537 | 14 | 519 | 2 |
| 60.0%/40.0% | 411 | 6 | 485 | 23 |
| 75.0%/25.0% | 398 | 11 | 372 | 1 |
| 100.0%/0.0% | 403 | 21 | 344 | 10 |

Table 2: This table has 6% cocamidopropyl betaine, 1.5% methyl acyl taurate, and 1.5% acyl isethionate. The synergy between acyl isethionate and methyl acyl taurate is evident at 9% surfactants at both 2.5 g and 5.0 g dilutions. The absolute numbers are slightly lower than in Table 1 because there is 3% anionic versus 4% in Table 1. The synergy can reliably be seen between acyl isethionate to methyl acyl taurate ratios of 60:40 and 40:60, as evident from the data in the above table.

TABLE 3
Table 3: 6% Surfactants actives with lauroyl methyl taurate
and cocoyl isethionate; 2:1 amphoteric to anionic

| Dilutions | | | | | | |
|---|---|---|---|---|---|---|
| Cocoyl methyl | | | | | | |
| taurate/Cocoyl | 10.0 g/250 mL, 1000 RPM | | 5.0 g/250 mL, 1000 RPM | | 2.5 g/250 mL, 1000 RPM | |
| Isethionate | Foam Volume | STD. | Foam Volume | STD. | Foam Volume | STD. |
| (wt. %/wt. %) | (mL) at 45 Sec. | Error | (mL) at 45 Sec. | Error | (mL) at 45 Sec. | Error |
| 0.0%/100.0% | 409 | 2 | 458 | 15 | Below Limit | Below Limit |
| 50.0%/50.0% | 569 | 21 | 468 | 14 | Below Limit | Below Limit |
| 100.0%/0.0% | 494 | 25 | 434 | 19 | Below Limit | Below Limit |

Table 3: In this example, there is 4% cocamidopropyl betaine, and 1% each of methyl acyl taurate and acyl isethionate. The synergy between methyl acyl taurate and acyl isethionate can be seen in formulations that are as low as 6% active surfactant as above. In this example, the standard error was high at the 5 g sampling relative to differential from the synergy, that a 10 g sample was required in order to show the synergy reliably. This is likely due to the low level of surfactant in the samples as noted, requiring additional product to increase the foam volume and subsequently the differentiation. The 2.5 g sample set was eliminated per our procedure due to its reading being under the 300 mL foam volume requirement.

TABLE 4
Table 4: 9% Surfactants actives with lauroyl methyl taurate
and lauroyl isethionate; 2:1 amphoteric to anionic

| Dilutions | | | | |
|---|---|---|---|---|
| Lauroyl methyl | 5.0 g/250 mL, | | 2.5 g/250 mL, | |
| taurate/Lauroyl | 1000 RPM | | 1000 RPM | |
| Isethionate | Foam Volume | STD. | Foam Volume | STD. |
| (wt. %/wt. %) | (mL) at 45 Sec. | Error | (mL) at 45 Sec. | Error |
| 0.0%/100.0% | 615 | 18 | 315 | 15 |
| 50.0%/50.0% | 661 | 1 | 332 | 12 |
| 100.0%/0.0% | 638 | 6 | 296 | 13 |

Table 4: The data above shows that formulations using lauroyl grades of both methyl acyl taurate, and acyl isethionate provide maximum lather boosting synergy at a ratio of 1:1. The data at the 2.5 g sample is relatively flat due to the low lather volume but is consistent with the invention.

TABLE 5
Table 5: 9% surfactant actives with cocoyl methyl taurate
and cocoyl isethionate; 2:1 Amphoteric to Anionic

| Dilutions | 5.0 g/250 mL, | | 2.5 g/250 mL, | |
|---|---|---|---|---|
| Taurate/ | 1000 RPM | | 1000 RPM | |
| Isethionate | Foam Volume | STD. | Foam Volume | STD. |
| (Wt. %/Wt. %) | (mL) at 45 Sec. | Error | (mL) at 45 Sec. | Error |
| 0.0%/100.0% | 334 | 5 | 315 | 8 |
| 50.0%/50.0% | 349 | 6 | 345 | 9 |
| 100.0%/0.0% | 335 | 2 | 316 | 6 |

Table 5: The data indicates that formulations using the cocoyl grades of methyl acyl taurate and acyl isethionate exhibit maximum synergy at a 1:1 ratio.

TABLE 6
Table 6: 9% Surfactant, 2:1 Amphoteric to Anionic

| | |
|---|---|
| Dilutions | |
| Taurate/ | 1.0 g/250 mL, 1500 RPM |

| Isethionate | Foam Volume | STD. |
|---|---|---|
| (Wt. %/Wt. %) | (mL) at 45 Sec. | Error |
| 0.0%/100.0% | 475 | 4 |
| 50.0%/50.0% | 632 | 1 |
| 100.0%/0.0% | 459 | 6 |

Table 6: The synergy seen with 2.5 g and 5 g samples at 1000 RPM in table 1 can also be seen with a 1 g sample at 1500 RPM. This sampling methodology has been shown to increase the lather generation and subsequently the differentiation between samples in samples sets.

TABLE 7
Table 7: 9% Surfactant, 1.5:1 Amphoteric to Anionic

| | |
|---|---|
| Dilutions | |
| Taurate/ | 1.0 g/250 mL, 1500 RPM |

| Isethionate | Foam Volume | STD. |
|---|---|---|
| (Wt. %/Wt. %) | (mL) at 45 Sec. | Error |
| 0.0%/100.0% | 420 | 2 |
| 50.0%/50.0% | 453 | 10 |
| 100.0%/0.0% | 427 | 10 |

Table 7: The synergy exists at 1:1 when the ratio of amphoteric to anionic is altered. The expression is lessoned in this formulation thus requiring the high shear parameters and 1 g of product to show differentiation.

### Example C

Compositions, consistent with this invention and comprising amine oxide, were made by mixing the following ingredients.

| Chemical | Active % |
|---|---|
| DI Water | Q.S. |
| Synthalen W2000 | 0.60 |
| Sodium Cocoyl Isethionate | 1.50 |
| Sodium Methyl Lauroyl Taurate | 1.50 |
| Stearic Acid | 0.05 |
| Glycerin | 1.00 |
| Cocoamidopropylamine Oxide | 6.00 |
| Tetrasodium EDTA | 0.05 |
| Phenoxyethanol | 0.60 |
| Iodopropyl Butyl Carbomate | 0.007 |
| Sodium Hydroxide | 0.016 |

1. A isotropic liquid composition comprising:
1) 0.1 to 8%, by weight of acyl isethionate;
2) 0.1% to 8%, by weight of methyl acyl taurate comprising lauroyl methyl taurate;
3) 0.0 to 15%, by wt weight of an amphoteric/zwitterionic surfactant comprising cocamidopropyl betaine;
4) 0.0 to 8.0%, by weight nonionic surfactant,
with the proviso that sum total of all surfactant is less than 20% by weight and the composition does not simultaneously comprise 0.01% by weight or more nonionic surfactant and 0.1% by weight or more amphoteric surfactant, including betaine comprising cocamidopropyl betaine and/or zwitterionic surfactant, and does not simultaneously comprise 0.0% by weight amphoteric and/or zwitterionic surfactant and 0.0% by weight nonionic surfactant, and wherein the ratio of methyl acyl taurate to acyl isethionate is surfactant is between 1.25:1 to 1:1.25 and further wherein the composition comprises 0 to 15% by weight glycerol.

2. The composition according to claim 1 wherein the composition has a pH from 5.0 to 7.4.

3. The composition according to claim 1 wherein the amphoteric surfactant is cocamidopropylbetaine.

4. The composition according to claim 1 wherein amphoteric, zwitterionic and/or nonionic to anionic are at a weight ratio from 1:1 to 4:1.

5. The composition according to claim 1 wherein nonionic or amphoteric and/or zwitterionic to anionic are at a weight ratio of 1:1 to 3:1 and the composition DH is 6.0 to 7.3.

6. The composition according to claim 1 wherein the ratio of amphoteric, zwitterionic and/or nonionic to anionic surfactant is 1.8:1 to 2.2:1 and the composition pH is 5.0 to 7.4.

7. The composition according to claim 1 wherein the pH is 6.3 to 7.3.

8. The composition according to claim 1 wherein amphoteric and/or zwitterionic surfactant make up from 0.0 to less than 0.1% by weight of the composition and nonionic surfactant is present at an amount from 0.02 to 8% by weight of the composition.

9. The composition according to claim 1 wherein nonionic surfactant is present at an amount from 0.001 to 7% by weight of the composition.

10. The composition according to claim 1 wherein nonionic surfactant is cocoamidopropyl amine oxide.

11. The composition according to claim 1 wherein the composition comprises less than 0.2% by weight sulfate based surfactant or no sulfate based surfactant.

12. A process of preparing p composition according to claim 1 wherein the process comprises:
i. mixing water and a structuring polymer, to produce a mixture and heating the mixture to about 75° C. (70-80° C.);
ii. adding isethionate and taurate to the heated mixture and mixing until the isethionate and taurate are dissolved;
iii. cooling the mixture to 55° C. and adding amphoteric, zwitterionic and/or nonionic surfactant; and
iv. adjusting mixture pH to 5.0 to 7.4
v. recovering composition,
wherein the structuring polymer comprises polysaccharide, polyacrylate or a mixture thereof and the composition comprises less than 0.2% by weight sulfate based surfactant.

13. (canceled)

14. The composition according to claim 1, wherein the composition comprises less 3.0% by weight betaine or no betaine.

15. The composition according to claim 1 wherein the composition comprises from 0.5 to 6% or from 1 to 4% by weight acyl isethionate.

16. The composition according to claim 1 wherein the composition comprises from 0.5 to 6% or from 1 to 4% by weight of methyl acyl taurate comprising lauroyl methyl taurate.

17. The composition according to claim 1 wherein the composition comprises from 0.1 to 15% or from 0.5 to 10% or from 1 to 8% by weight of an amphoteric/zwitterionic surfactant comprising cocamidopropyl betaine.

18. The composition according to claim 1 wherein the composition comprises from 0.001 to 7% or from 1 to 6% by weight nonionic surfactant.
